(19) United States
(12) Patent Application Publication
(10) Pub. No.: US 2024/0272182 A1
(43) Pub. Date: Aug. 15, 2024

(54) BROAD SPECTRUM DETECTION KITS

(71) Applicant: Veriteque USA, Inc., Carson City, NV (US)

(72) Inventor: Christian Loane, Carson City, NV (US)

(21) Appl. No.: 18/568,467

(22) PCT Filed: Jun. 9, 2022
(86) PCT No.: PCT/US2022/032910

§ 371 Date: Dec. 8, 2023

Related U.S. Application Data
(60) Provisional application No. 63/208,849, filed on Jun. 9, 2021.

(51) Int. Cl. G01N 33/94; G01N 33/52

(52) U.S. Cl. CPC G01N 33/948; G01N 33/52; G01N 33/946

(57) ABSTRACT

Detection kits for identifying the presence of a drug are provided. In one exemplary implementation, the kit includes a dry colorimetric reagent and a delivery device containing a solvent or solvent mixture including the solvent. The delivery device is configured to deliver a portion of the solvent or solvent mixture to a target residue to form a sample residue. When at least a portion of the sample residue is brought into contact with the mixture, the mixture undergoes a chemical reaction when the sample residue contains a drug, in which the chemical reaction produces a visible color change that is indicative of the presence of the drug within the sample residue. Methods of manufacturing a detection kit are also provided.

### CROSS-REFERENCE TO RELATED APPLICATIONS

This application claims priority to U.S. Provisional Patent Application No. 63/208,849, filed Jun. 9, 2021, and entitled “Broad Spectrum Field Kit,” the disclosure of which is incorporated herein by reference in its entirety.

### FIELD

Detection kits for identifying the presence of a broad spectrum of drugs and methods of manufacturing and using the same are provided.

### BACKGROUND

The trafficking and smuggling of contraband and narcotics across borders is an immense global problem. On a different scale, this issue also exists within prison and correctional facilities. Across borders, large scale carriage systems are available e.g. backpacks, boats, cars, planes. Across prison security check points, the contraband is typically hidden within smaller more intricate “innocent” products. One trend that has gained momentum in the past 5 to 10 years is smuggling narcotics impregnated in paper products or mixed with other consumer products (e.g., e-cigarette formulations).

Narcotic test kits are commercially available. For example, some commercially available test kits contain liquid reagent dropper bottles, powder reagents, and alcohol swabs, whereas others contain aerosolized liquid reagent spray cans or crushable glass ampoules filled with liquid reagents. Unfortunately, these commercial test kits are typically designed to detect limited sample forms of drugs and in only a certain number of drug classes. Further, these kits suffer from a variety of manufacture and end use problems, for example: (i) liquid dropper bottles, breakable glass or plastic ampoules, blister packs or pressurized aerosol spray cans filled with hazardous liquid reagents, (ii) the presence of hazardous liquid reagents pose problems with manufacturing and exposure limitations, storage and handling, strict packaging requirements and significant shipping restrictions; (iii) the volume or quantity of liquid reagents consumed during one single test is excessive and wasteful, thereby adding to costly, bulky and often overly complicated device design and packaging; (iv) during use, operators may be exposed to sharps and hazardous liquid reagent splash or overspray; and (v) to complete a single test, the device can involve multi-step operations.

Accordingly, there remains a need for improved detection kits that address current issues with narcotic test kits.

### SUMMARY

Aspects of the current subject matter relate to detection kits capable of detecting a wide variety of drug classes. In some variations, one or more of the following features may optionally be included in any feasible combination.

An objective of the disclosed implementations is to design a low cost, mass producible, field deployable, spot test kit which will facilitate identification of drugs from most drug classes. Such drugs can be provided as discrete powers or liquids, concealed as contraband within articles, concealed within commercial formulations as legitimate additives to the formulation, or as an illicit adulterant to both commercial and/or clandestine formulations.

It is a further objective of the disclosed implementations to provide a kit with a simple, sample collection technique, which minimizes the destruction and contamination of surfaces, forensic residue, crimes scenes and the like, thus greatly aiding the forensic process.

It is a further objective of the disclosed implementations to design a kit, constructed of simple absorbent material (e.g., cotton swab products or fibrous wipe(s)), with all color change reagents supplied in simple hermetically sealed closures, constructed of non-hazardous materials and components.

Still a further objective of the disclosed implementations is to design a portable test kit, which has low cost, mass manufacture capability, in the order of millions of units per annum, while achieving commercial kit shelf life, in the order of several years and a reduced false alarm rate(s).

In one exemplary implementation, the kit includes a dry colorimetric reagent and a delivery device containing a solvent or solvent mixture including the solvent. The delivery device is configured to deliver a portion of the solvent or solvent mixture to a target residue to form a sample residue. When at least a portion of the sample residue is brought into contact with the mixture, the mixture undergoes a chemical reaction when the sample residue contains a drug, in which the chemical reaction produces a visible color change that is indicative of the presence of the drug within the sample residue.

In some implementations, the dry colorimetric reagent includes bismuth nitrate, potassium iodide, and an acid. In certain implementations, the acid can be an organic acid. The organic acid can include tartaric acid, ascorbic acid, oxalic acid, toluenesolfonic acid, benzoic acid, citric acid, and any combination thereof. In other implementations, the acid can be an inorganic acid.

In some implementations, the mixture can be configured to undergo chemical reaction with the drug in the form of liquids, gels or solid powders that are pure or admixed with cutting agents.

In some implementations, the drug can be at least one of an amphetamine, cocaine, cathinone, heroin, lysergic acid diethylamide, nicotine, a synthetic cannabinoid, and a fentanyl analogue.

In some implementations, the detection kit can include a substrate having the dry colorimetric reagent disposed on at least a portion thereof. The at least a portion of the substrate with the dry colorimetric reagent can form a reaction zone, in which the chemical reaction occurs at the reaction zone. The substrate can have a variety of configurations. In some implementations, the substrate can be formed of at least one of fibers or polymers. In other implementations, the substrate can be a paper card, a paper sheet, a synthetic paper, or chromatography paper.

In some implementations, the substrate and the delivery device can each be enclosed and separated from each other in respective containers.

In some implementations, the detection kit can include a packet that can be configured to selectively house the substrate and the delivery device. In some implementations, the packet can be hermetically sealed.

The delivery device can have a variety of configurations. In some implementations, the delivery device can include an absorbent material. In some implementations, the delivery device can be a cotton swab that absorbs the solvent or solvent mixture. In some implementations, the delivery device can be a snap cotton swab that can selectively store the solvent or solvent mixture in a shaft.

Methods of manufacturing a detection kit are also provided. In one exemplary implementation, the method includes preparing an ink formulation that includes a colorimetric reagent and at least one solvent, applying, by a printing process, the ink formulation on a least a portion of a substrate, and drying the ink formulation to form a dry colorimetric reagent on the substrate. The dry colorimetric reagent is configured to undergo a chemical reaction with a sample residue having a drug that produces a visible color change colorimetric indication.

In some implementations, the dry colorimetric reagent includes bismuth nitrate, potassium iodide, and an acid. In certain implementations, the acid can be an organic acid. The organic acid can include tartaric acid, ascorbic acid, oxalic acid, toluenesolfonic acid, benzoic acid, citric acid, and any combination thereof. In other implementations, the acid can be an inorganic acid.

In some implementations, the dry colorimetric reagent on the substrate forms a reaction zone, wherein the chemical reaction occurs at the reaction zone.

In some implementations, the method can include storing a solvent or solvent mixture including the solvent within a delivery device, in which the delivery device can be configured to deliver a portion of the solvent or solvent mixture to a target residue to form the sample residue. The storing the solvent or solvent mixture can include dipping the delivery device into a solution that can include the solvent or solvent mixture. The solvent mixture can include a denatured alcohol and water in a ratio of 50:50, in which the denatured alcohol can be present in the solvent mixture at a volume of about 0.05 mL to about 0.2 mL.

In some implementations, preparing the ink formulation can include dissolving a first reagent of the colorimetric reagent and the at least one acid into a first solution, dissolving a second reagent of the colorimetric reagent in a second solution, and mixing the first and second solutions together to form the ink formulation. The first solution can include denatured alcohol, water, and glycerol. The second solution can include water.

The substrate can have a variety of configurations. In some implementations, the substrate can be a bibulous carrier material. In some implementations, the substrate can be a cellulose chromatography paper.

In some implementations, the printing process can be one of spray printing, dip printing, flexography, offset screen printing, lithography, digital printing, or gravure printing.

In some implementations, the printing process can be a flexographic printing process.

The details of one or more variations of the subject matter described herein are set forth in the accompanying drawings and the description below. Other features and advantages of the subject matter described herein will be apparent from the description and drawings, and from the claims. The claims that follow this disclosure are intended to define the scope of the protected subject matter.

### BRIEF DESCRIPTION OF THE DRAWINGS

The accompanying drawings, which are incorporated into and constitute a part of this specification, show certain aspects of the subject matter disclosed herein and, together with the description, help explain some of the principles associated with the disclosed implementations. In the drawings:

FIG. 1 illustrates a delivery device in the form of a swab made in accordance with the disclosed implementations;

FIG. 2A illustrates a delivery device in the form of a cotton tipped snap swab made in accordance made in accordance with the disclosed implementations, showing the cotton tipped snap prior to snapping;

FIG. 2B illustrates the cotton tipped snap swab after it is snapped, thereby releasing solvent into the cotton tip;

FIG. 3A illustrates a delivery device in the form of a fiber tipped pop swab made in accordance with the disclosed implementations, showing the swab prior to squeezing;

FIG. 3B illustrates the fiber tipped pop swab after it is squeezed, thereby releasing solvent into the fiber tip;

FIG. 4 illustrates a sheet of dry chemical reagents in accordance with the disclosed implementations;

FIG. 5 illustrates a tableted form of dry chemical reagents in accordance with the disclosed implementations;

FIG. 6 illustrates an exemplary packaged detection kit in accordance with the disclosed implementations, the detection kit having a substrate with a printed reaction zone and a deliver device;

FIG. 7 illustrates the substrate and the swab of FIG. 6, showing the delivery device with a sample residue being pressed onto the printed reaction zone of the substrate to determine the presence of a drug within the collected sample residue; and

FIG. 8 illustrates a manufacturing process in accordance with the disclosed implementations.

When practical, similar reference numbers denote similar structures, features, or elements.

### DETAILED DESCRIPTION

Provided herein, inter alia, are kits and methods for detecting most drugs classes and street drugs of abuse, including but not limited to: synthetic cannabinoids, cathinones, amphetmines, cocaine, lysergic acid diethylamide (LSD), nicotine, heroin, fentanyl analogues, and the like. The present disclosure relates to a portable detection kits capable of identifying the presence of drug classes as powder, solids and liquids on surfaces and/or hidden as contraband within different matrices and mixed into commercial formulations as part of a manufactured article or, illicitly added to a formulation, thereby constituting a counterfeit or clandestine article.

The present disclosure provides a design for a low cost, mass producible, field deployable, spot detection kit which will facilitate identification of most drug classes, both conventional and evolving designer drugs, from many surfaces, including but not limited to skin, metals, glass and fabrics such as paper, cloth and organic polymer substrate, while minimizing operator exposure to hazardous chemicals.

The detection kit described herein generally includes at least one dry reagent (e.g., dry colorimetric reagent(s)). The term “dry” when used to modify “reagent” or “colorimetric reagent,” refers to the reagent or the colorimetric reagent having a moisture content of 5 weight % or less. For example in some implementations, the moisture content can be from 4 weight % or less, 3 weight % or less, 2 weight % or less, or 1 weight % or less. In some implementations, the moisture content can be less than 1 weight %. In some implementations, the at least one dry reagent or the dry colorimetric reagents(s) can be in the form of a powdered, granular, solid crystalline structure(s).

The dry reagent can be provided in any suitable modality for end use which is simple to mass produce and achieves long term shelf life, ease in shipment and carriage and simplicity in use and disposal. By way of example and without limitation such modalities may include a printed card, paper, plastic, strips, and the like. In such implementations, the detection kit can also include a delivery device with a solvent or solvent mixture. The solvent(s) may be selected from both aqueous and non-aqueous species. In some implementations, the solvent can be water, isopropyl alcohol, ethanol, or denatured alcohol. In one implementation, the solvent mixture can include an alcohol (e.g., denatured alcohol):water co-solvent mix in the ratio of 50:50. In such implementations, the volume the alcohol is present in the solvent mixture can be at a volume of about 0.05 mL to about 0.2 mL. In some implementations, the delivery device can include an absorbent material.

In some implementations, a detection kit, without limitation, may be provided for use in any combination as separate dry powder(s) and solvents(s) or mixed together in any combination as dissolved solution(s) or homogenized suspension(s) and be delivered for use without limitation in any combination of packaging or containers or support articles, wherein the physical implementation of the kit and its components are provided in individual hermetically sealed light, air and moisture proof packaging for ease in transport, handling and long term storage prior to use.

In some implementations, the kits and methods described herein provide for a delivery device in the form of a pre-wetted swab. The co-solvent, which is to be absorbed into the pre-wetted swab, may be prepared by mixing (i) dentatured alcohol with (ii) water, to form the a swab solvent. The process of mixing the swab solvent can be achieved by placing the correct mass of each individual solvent into any suitable beaker or screw top bottle and with impeller or bench top bottle roller, mixing for some period of time, to thereby form a swab solvent. In one implementation, the swab solvent is homogenous.

In some implementations, the kits and methods described herein provide for a pre-wetted swab. The swab can be a single tipped cotton q-tip. The swab can be of any dimension or size. The swab shaft may be made of any suitable material, for example without limitation wood, polypropylene, pvc and the like. The q-tip fibrous matrix can be constructed of any suitable material, for example and without limitation felt, cotton, natural and synthetic fibers. Exemplary swab design is a 3 mm (O.D.) white polypropylene shaft×80 mm (L) with white natural fiber cotton tip. The process of pre-wetting the swab can be achieved by any suitable mechanized automated process and manually by hand. For example, the cotton q-tip can be immersed into or contacted with a volume of the swab solvent, in excess of 0.2 mL thus achieving correct wetting.

In implementations, the pre-wetted solvent swab, once produced, would be inserted into a package in a suitable format for safe storage, shipping and later use, for example form fill sealed into hermetically sealed light, air and moisture proof packaging.

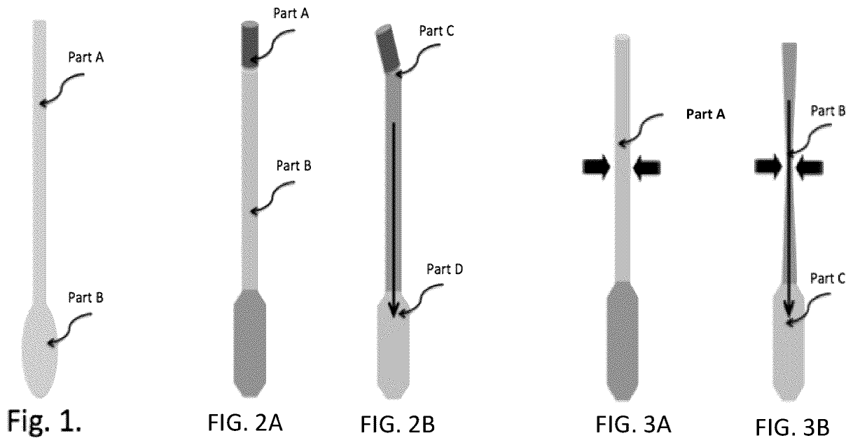

In accordance with implementations of the present disclosure, and without limitation, in one implementation, the delivery device is in the form of a pre-wetted cotton swab, as illustrated in FIG. 1. This modality allows for excellent residue dissolution and pickup from surfaces, does not destroy surface coatings or forensic residue, may be applied to human skin and clothing fabrics, and has no hazardous chemical features. The cotton swab includes a handle (Part A) and a tip or head (Part B). The pre-wetting can be achieved by simple dip and/or rapid immersion of the cotton swab matrix into large volume pre-mixed solvent or solvent mixture, vat or micro-jet spray or similar. Pre-wetting can be a fully automated process utilizing conventional conveyor, hopper, spray machinery.

In some implementations, the kit and methods described herein provide for a snap or pop swab with swab shaft containing the swab solvent and swab tip containing pre-impregnated and dried chemical change reagents. The dried chemical change reagents in combination form a dried colorimetric reagent. The swab shaft is solvent can be filled by any commercial process well known in the art and the dry powder reagents can be dissolved applied, e.g., via spray-dry or dip-dry, to swab tip. Once produced, the swabs would be inserted into a package in a suitable format for safe storage, shipping and later use, for example form fill sealed into hermetically sealed light, air and moisture proof packaging.

In use, once the target residue or surface to be tested has been located, the snap or pop swab is removed from the shipping packaging, with the operator holding the shaft of the swab, the shaft is snapped or popped to release solvent into the tip, at which tip the swab tip is pressed onto target residue for 10 seconds, thereby allowing dissolution of residue and color reagents, facilitating a chemical reaction and presumptive color indication. This visual color change may be viewed in the swab tip once swab tip is removed from residue/surface.

In another implementation and without limitation, the delivery device is in the form of a “snap cotton swab,” as illustrated in FIGS. 2A-2B. The hollow swab handle (Part A) is pre-filled in fully automated commercial fill processes with a solvent or a solvent mixture, as shown in FIG. 2A. The tip of the swab shaft (Part A) has etched grooves applied during swab manufacture, which is easily snapped (Part C) between thumb and forefinger, releasing the shaft contents down and into the cotton tip (Part D), as shown in FIG. 2B. In some implementations, the delivery device can also include the homogenized dry reagent powders. For example, the tip of the snap can have the homogenized dry reagent powders lodged within the tip fiber matrix. The tip of the snap can have the dry reagent powders spray-n-dried or dip-n-dried into tip fiber matrix.

In yet another implementation and without limitation, the delivery device is in the form of a “pop cotton swab,” as illustrated in FIGS. 3A-3B. The hollow flexible swab handle (Part A) is pre-filled in fully automated commercial fill processes with a solvent or solvent mixture, as illustrated in FIG. 3A. The handle of the swab handle is easily squeezed (Part B) and small closure contained with hollow shaft handle broken or “popped” between thumb and forefinger, releasing the shaft contents down and into the cotton tip (Part C), as illustrated in FIG. 3B. In some implementations, the delivery device can also include the homogenized dry reagent powders. The tip of the snap can have the homogenized dry reagent powders lodged within the tip fiber matrix. The tip of the snap was, as described in the preceding sections of this disclosure can have the dry reagent powders spray-n-dried or dip-n-dried into tip fiber matrix.

In some implementations, the kit and methods described herein provide for a pressure formed or pressed article, for example without limitation a tablet. A dry powder formulation can contain all dry powder reagent(s) (e.g., dried colorimetric agents), tablet excipients and release agents, which enable the formation of a tablet structure of any size or dimension. The tablet, once produced, would be inserted into a package in a suitable format for safe storage, shipping and later use, for example form fill sealed into hermetically sealed light, air and moisture proof packaging.

In use, once the target residue or surface to be tested has been located, a pre-wetted solvent swab, as previously described, and the tablet are removed from the shipping packaging, with the operator holding the shaft of the swab tip, the swap tip is pressed onto target residue for 10 seconds, thereby allowing dissolution of residue, facilitating a chemical reaction and presumptive color indication. Presumptive indication (described previously) may be viewed in the swab tip once swab tip is removed from residue/surface. This visual color change may be viewed in the swab tip once swab tip is removed from residue/surface.

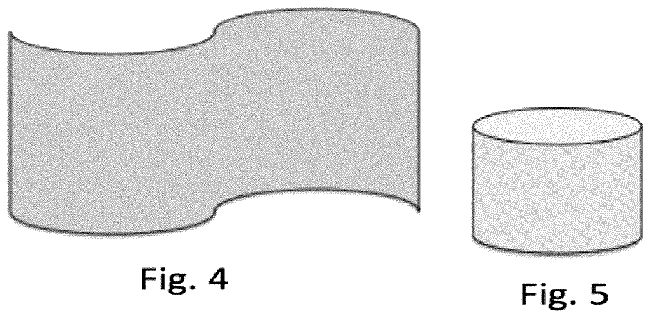

In other implementations, the detection kit can include at least one dry reagent in the form of a pressed sheet, as illustrated in FIG. 4, or in the form of a pressed tablet, as illustrated in FIG. 5. In such implementations, binders and release agents can be combined with the at least dry reagent.

In implementations, the dry reagent(s) in combination with a solvent(s) undergo physico-chemical interaction with the functional groups attached to the molecular structure of a drug (e.g., conventional street drugs of abuse or designer drugs), thereby producing a colored reaction product, resulting in a visible color change also known as a presumptive colorimetric indication. For example, in use, an operator of the kit can take a delivery device and bring it into contact with a target residue to form a sample residue. Once the sample residue is obtained, the sample residue is brought into contact with the combination of the dry reagent(s). In instances, where a drug is present within the sample residue, the dry reagent and solvent undergo a chemical reaction that produces a produces a visible color change (e.g., an identifiable color change by a human eye) that is indicative of the presence of the drug within the sample residue. The drug to be detected may be present as solid particulate, fine powder, gels, and liquids. Further, the drug may be pure or admixed with cutting agents.

In some implementations, the dry reagent(s) can include (a) bismuth nitrate, (b) potassium iodide and (c) acid(s) (e.g., organic or inorganic acids), which in combination form a dry colorimetric reagent. The drug in the target residue(s) may be detected in molecular and ionic form, as the free base or acid salt(s), in physical form as solids, liquids and gels, from (i) surfaces, (ii) impregnated into a polymeric matrices, (iii) or mixed into a commercial or clandestine liquid formulations, by using any combination(s) of (a)-(c) and a delivery device (e.g., a pre-wetted solvent swab) to facilitate a presumptive colorimetric indication, thus identifying the presence of narcotics in (i)-(iii). Non-limiting examples of suitable acids include tartaric acid, ascorbic acid, oxalic acid, toluenesolfonic acid, benzoic acid, and citric acid. In one implementation, the acid is citric acid.

In implementations, the kits, without limitation, may be provided for use in any combination as separate dry powder(s) and solvents(s) or mixed together in any combination as dissolved solution(s) or homogenized suspension(s) and be delivered for use without limitation in any combination of packaging or containers or support articles. The physical implementation of the kit and its components can be provided in individual hermetically sealed light, air and moisture proof packaging for ease in transport, handling and long term storage prior to use.

Contrary to current approaches for the presumptive identification of most drugs classes, conventional street drugs of abuse and designer drugs, it has been discovered that selected drug indicating formulations may be printed to a substrate, e.g., by the use of conventional mass production printing equipment, using conventional tooling. Once printed, the resulting structure includes a dried colorimetric reagent(s) that can be used to detect a drug in a sample residue. The printed substrate can be packaged in various combinations with a delivery device (e.g., a pre-wetted swab), which together provides a selective, cheap, mass producible, portable detection kit for the presumptive identification of most drugs classes, present as either solid or liquid residue or hidden with commercial and/or illicit product.

The detection capability afforded by the present detection kits is single step operation. Further, an advantage of present kits disclosed herein is increased operator safety when using the kit, which allows a test to be carried out without the need for touching suspect residue or articles (e.g., target residue or article). Other advantages of the present kits disclosed herein include, for example, no spoilage, damage and/or contamination of residue and surfaces, thus preventing destruction of forensic evidence.

In some implementations, the detection kits can include a dry colorimetric reagent(s) and a delivery device containing a solvent or solvent mixture including the solvent. The delivery device can be configured to deliver a portion of the solvent or solvent mixture to a target residue to form a sample residue. In use, when at least a portion of the sample residue is brought into contact with the mixture, the mixture undergoes a chemical reaction when the sample residue contains a drug. The chemical reaction can produce a visible color change that is indicative of the presence of the drug within the sample residue. The target residue can be taken from many different surfaces, including but not limited to skin, metals, glass and fabrics such as paper, cloth and organic polymer substrate, while minimizing operator exposure to hazardous chemicals. In one implementation, the dry colorimetric reagent(s) can include bismuth nitrate, potassium iodide, and an acid.

In some implementations, the dry colorimetric reagents can be deposited on a least a portion of a substrate. In certain implementations, the at least a portion of the substrate with the dry chemical reagents forms a reaction zone, in which the chemical reaction occurs at the reaction zone. In general, a suitable substrate can be any material which allows an ink formulation (e.g., a colorimetric reagent(s) and at least one solvent) to be bound to and dried on, its surface. Non-limiting examples of suitable substrates include natural and/or synthetic fiber/polymer paper products. As such, suitable substrates can include at least one of fibers or polymers. In some implementations, the substrate can include a paper card, a paper sheet, a synthetic paper, or chromatography paper. Exemplary substrates include 200 to 300 gsm, 10-14PT, 20 mm paper product supplied as Technicote, Fasson, Avery, Whatmann or similar products. One exemplary substrate is Whatman 3030-700 Cellulose Chromatography Paper, Grade 3MM Chr Roll, 23 cm×100 m. Another exemplary substrate is a bibulous carrier material.

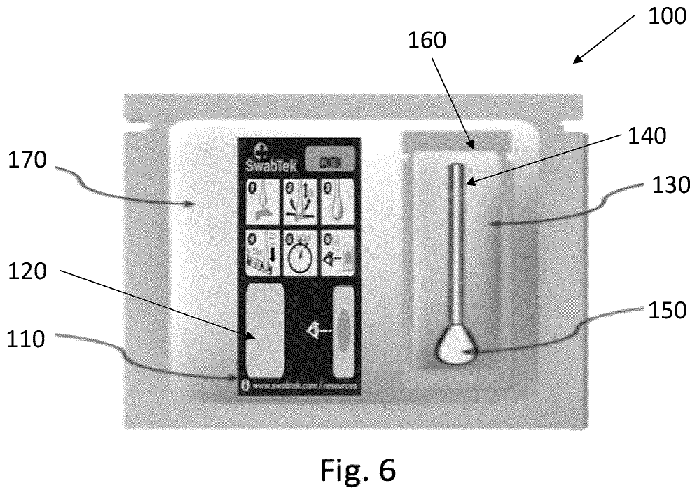

For example, as illustrated in FIG. 6, a detection kit 100 includes a substrate 110 (e.g., Test Card) with printed dry colorimetric reagent(s) 120 deposited on a portion the substrate, to thereby from a reaction zone. Further, the detection kit includes a delivery device 130 that is in the form of a pre-wetted cotton swab, which includes a handle 140 and a tip or head 150. As noted above, the pre-wetting can be achieved by simple dip and/or rapid immersion of at least the cotton swab tip or head 150 into large volume pre-mixed solvent or solvent mixture, vat or micro-jet spray or similar. Further, in some implementations, the substrate and the delivery device are each enclosed and separated from each other in respective containers. For example, as shown in FIG. 6, the delivery device 130 can be contained within a sleeve 150, and the substrate 110 and the sleeved delivery device can be housed within a packet 170. In certain implementations, the sleeve 150 and/or the packet 170 can be hermetically sealed.

In some implementations, the delivery device can be packaged in moisture and UV resistant packages (e.g., a sleeve) prior to use. In one implementation, the packaging could be a tare open, form, fill and seal sachet. The sachet can be constructed from commercially available materials, for example, a Paper/PET12 um/AL7 um/PE50 product.

In some implementations and without limitation, the detection kit can be carried in a pocket, belt case, glove box, brief case, etc. Once the suspect residue or surface to be tested, has been located, both the packet 170 and the sleeve 150 are opened. The pre-wetted swab is removed from the shipping packaging, with the operator holding the shaft of the swab, and it is firmly pressed and dabbed into or across the target residue or surface, for approximately 10 seconds. This is achieved without need for operator to touch suspect residue or surface.

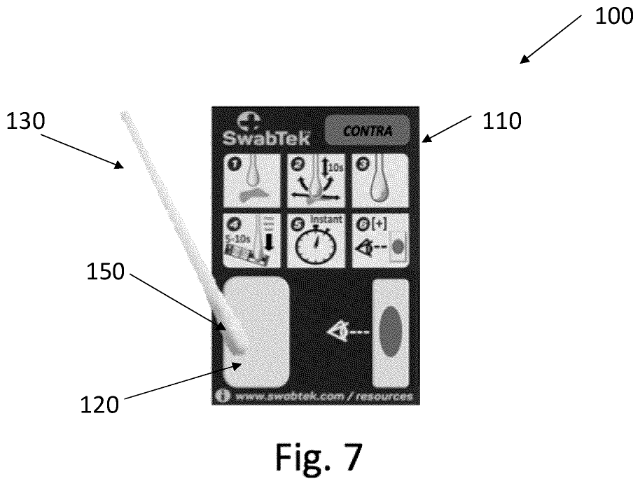

The sampled swab is then transferred to the printed substrate and the sampled swab tip is pressed down into the printed test reaction zone on the printed substrate for 5 seconds, as shown in FIG. 7. During the 5 seconds of contact, the swab solvent/dissolved residue, collectively referred to herein as sample residue, wicks out from the swab absorbed into the matrix of the substrate (e.g., a fiber matrix), allowing the sample residue and dry printed reagents to mix, thus facilitating a chemical reaction and visible presumptive color indication for the presence of conventional street drugs of abuse and designer drugs within the sample residue. The swab is then removed from the surface of the substrate, allowing the operator to visually observe a presumptive color indication. This is all achieved without need for the operator to come into contact with dry powder reagents(s), solvent(s) and target residue(s).

In implementations, the method provides that the operator views both the swab tip and the substrate for visual color changes, and thus a colorimetric indication of the presence of a drug within the sample residue. By way of example, (A) Positive presumptive colorimetric indication for the presence of conventional street drugs of abuse and designer drugs is rapid/instant formation of obvious dark orange to red to pink—pink color formation, (B) Negative result—no color change—bleaches white color—grey to black outline. In certain implementations, positive color indications can occur and be visible from 1 second onwards after the sample residue is in contact with the printed reaction zone of the printed substrate.

Exemplary results from using the kits as described herein can include, where negative is indicative of no drug and positive is indicative of drug within the sample residue:
NEGATIVE RESULT: no color change of yellow/gold reaction zone.
NEGATIVE RESULT: white or bleached zone.
NEGATIVE RESULT: white or grey or black hue.
POSITIVE RESULT: Dark orange, red or pink hue.
   In some implementations, the visual indications can appear as a spherical shape as the solvent wicks outwards and absorbs radially into the test card printed reaction zone.

In some implementations, the methods described herein provide for the manufacturing of the present detection kits. For example, in general, the method can include preparing an ink formulation that includes a colorimetric reagent and at least one solvent, applying, by a printing process, the ink formulation on a least a portion of a substrate; and drying the ink formulation to form a dry colorimetric reagent on the substrate. Non-limiting examples of suitable printing processes include spray printing, dip printing, flexography, offset screen printing, lithography, digital printing, and gravure printing.

The ink formulation can be prepared using a variety of different methods. For example, dissolving a first reagent of the colorimetric reagent into a first solution; dissolving a second reagent of the colorimetric reagent in a second solution; and mixing the first and second solutions together to form the ink formulation. The first reagent can include bismuth nitrate and at least one acid (e.g., citric acid). The second reagent can include potassium iodide.

In some implementations, dissolving the first reagent of the colorimetric reagent into a first solution can include, by way of example, mixing (a) bismuth nitrate with (c) citric acid and dissolving (a)+(c) mix in a solution of (i) alcohol+(ii) water+(iii) glycerol, to form a “Solution I”. Solution I may be prepared in the ratio of (a) bismuth nitrate (1.55 grams) (c) citric acid (30 grams) (i) de-natured alcohol (25 grams) (ii) water (100 grams) and (iii) glycerol (10 grams). The process of mixing and dissolution can be achieved by placing the correct mass of each dry powder and each solvent, into any suitable beaker or screw top bottle and with impeller or bench top bottle roller mixing for some period of time, to thereby form Solution I. In certain implementations, Solution I is a homogenous solution.

In some implementations, dissolving a second reagent of the colorimetric reagent in a second solution can include, by way of example, mixing (b) potassium iodide with (ii) water, to form a “Solution II”. Solution II may be prepared in the ratio of (b) potassium iodide (9 grams) with (ii) water (30 grams). The process of mixing and dissolution can be achieved by placing the correct mass of each dry powder and each solvent, into any suitable beaker or screw top bottle and with impeller or bench top bottle roller mixing for some period of time, to thereby from Solution II. In certain implementations, Solution II is a homogenous solution.

Once Solution 1 and Solution 2 are prepared, the ink formulation can be prepared, for example, by mixing Solution I with Solution II. The ink formulation may be prepared by simply pouring the full volume of Solution I and the full volume of Solution II into any suitable beaker or screw top bottle and with impeller or bench top bottle roller mixing for some period of time (e.g., approximately 1 hour prior to printing), thus producing the ink formulation.

Once the ink formulation is prepared, it can then be applied, by a printing process, to at least a portion of a substrate. For example, the printed substrate, also referred herein as a Test Card or Test Strip, can be prepared by a suitable industrial scale, mass output printing process. This can be achieved in any combination with spray and dry, dip and dry, flexography, offset screen, lithography, digital printing and gravure printing systems. An exemplary printing system is Flexography, which allows a wide web, inter-changeable, multiple ink, roll to roll, guillotine process in a single step. The flexographic press is run at printing speeds in conjunction with heater drying to achieves a final printed product. Without limitation a suitable machine is a Mark Andy Flexographic press. In such implementations, the ink formulations described herein are designed for a fully automated industrial scale printing method, and therefore are able to withstand the harsh environment of industrial tooling and process. The ink formulations described herein can provide a suitable liquid viscosity for incorporation into conventional printer tooling, adequate adhesion to the anilox rollers achieving sufficient volumetric laydown on to the “web” of a substrate (e.g., paper substrate) and (iii) offers rapid solvent flash off drying prior to final web Test Card guillotine cutting.

In implementations, the printed Test Card, once produced, can then be inserted into a package in a suitable format for safe storage, shipping and later use. For example, the printed Test Card can be inserted in a package via a form fill seal process into hermetically sealed light, air and moisture proof packaging.

In implementations, the methods described herein provide for a Flexography method of kit manufacture. The disclosure also provides for a modification of the Flexography tooling, which allows “excess volume” of “Ink” lay down (print), while maintaining adequate resolution and drying of printed image. The “excess volume” “Ink” lay down may be achieved by any combination of (i) large cavity anilox, (ii) screen hatching and (iii) use of non-conventional printing substrates in place of the screen. The non-conventional substrates are any natural or synthetic foam and/or fibrous material, able to be laminated to an adhesive backing and allows semi-permanent adhesive fixing to the flexible screen material and/or print roller tooling of the Flexographic press. Exemplary materials, without limitation are, adhesive backed and flexible 3-5 mm thick industrial foams, Velcro, fiber matting and the like. These materials offer desirable pickup of “Ink” from the anilox and high percentage transfer of this “Ink” volume web.

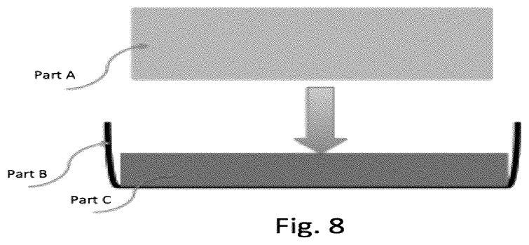

FIG. 8 illustrates another exemplary manufacturing format in accordance with the present teachings, which involves a “dip-n-dry” process to form a pre-impregnated reagent bibulous carrier. In use, chromatography paper (e.g., whatman #1 CP), referenced as Part A in FIG. 8, is partially dipped into a reagent solution (Part C) held in a dipping tray (Part B). The reagent solution wicks and absorbs into the chromatography paper. The wetted paper is then removed from the dipping tray (Part B) for subsequent drying by hot air stream to form pre-impregnated reagent bibulous carrier. In such instances, the pre-impregnated reagent bibulous carrier can function as a Test Card or Strip as described herein.

### EXAMPLES

Implementations herein are further illustrated by the following examples and detailed protocols. However, the examples are merely intended to illustrate implementations and are not to be construed to limit the scope herein.

### Example 1

Pre-wetted swab: A pre-wetting a cotton Q-tip with 0.05 to 0.2 mL of a co-solvent mix with a Denatured alcohol (ethanol):Water (50:50)“Solvent 1” is produced. Individually packaging said swab into a hermetically form fill sealed Paper/PET12 um/AL7 um/PE50 sachet.

“Ink” production: The “Ink” is produced as follows: Solution I—may be prepared in the ratio of (a) bismuth nitrate (1.55 grams) (c) citric acid (30 grams) (i) de-natured alcohol (25 grams) (ii) water (100 grams) and (iii) glycerol (10 grams). Solution II—may be prepared in the ratio of (b) potassium iodide (9 grams) with (ii) water (30 grams). Finally, the printing “Ink” is prepared by adding Solution (I) to Solution (II) into a suitable beaker or plastic screw top bottle and utilising impeller or bench top roller, mix “Ink” solution for approximately 1 hour prior to printing step.

Substrate: 200 to 300 gsm, 10-14PT, 20 mm paper product supplied as Technicote, Fasson, Avery, Whatmann or similar products. An exemplary substrate Whatman 3030-700 Cellulose Chromatography Paper, Grade 3MM Chr Roll, 23 cm×100m.

Flexo press: A suitable 8 to 10 station Mark Andy flexographic press. High temperature air heaters. Maximum Anilox roll volume with fewer cells. Screen with maximum hatching design. Screen may be replaced with “novel” adhesive back “foam” and or “Velcro” structures to facilitate “Ink” lift and deposition to paper web. Web speed to minimum.

Test card printing, guillotine and packaging: The Test Card is printed by adding “Ink” to appropriate station in Flexo press. In combination with graphic art ink stations the Test Card graphics and the “Ink” reaction zone are printed onto the Paper web, hot air dried and guillotined into final desired shape. The final Test Card product is packaged in a sachet constructed from commercially available Paper/PET12 um/AL7 um/PE50 product, which is an extremely cheap, mass produced material.

### Example 2

### General Use of Kit:

Where a target residue is observed, both the pre-wetted swab and test card are removed from the packaging and held in the hand of the operator. The pre-wetted swab pressed into and dabbed onto physical residue for approximately 10 seconds. The sample swab is transferred and pressed firmly onto reaction zone printed on the test, for 5 seconds. After this time the operator lifts the swab to reveal the presumptive indication.

### Color Indications:

In accordance with the current invention and without limitation, example results from using the kit of the current invention are provided below: NEGATIVE: no color change of yellow/gold reaction zone. NEGATIVE: white or bleached zone. NEGATIVE: white or grey or black hue. POSITIVE: Dark orange, red or pink hue.

### Example 3

### Synthetic Cannabinoid JWH007 Impregnated Legal Document:

Where a suspect residue is observed, both the pre-wetted swab and test card are removed from the packaging and held in the hand of the operator. The Test card is placed underneath a suspect legal document, on a firm surface. The swab is repeatedly pressed down onto the legal document over top of the preprinted reaction zone of the Test Card. The document is wetted over top of the printed reaction zone. Then pressing the swab firmly down for 5 see, contacting swab/legal document/printed reaction zone, any narcotics present in that region of the legal document are dissolved and wicked into the printed reaction zone of the Test Card affording a visual deep orange presumptive indication for the presence of narcotics and in this case JWH007, impregnated into the document.

### Example 4

Nicotine in 0%/0 mg/mL Illicit e-Juice:

Where a suspect residue is observed, both the pre-wetted swab and test card are removed from the packaging and held in the hand of the operator. A single drop of the suspect e-juice is either applied directly to the test card printed reaction zone or using swab to collect sample from e-juice bottle or paraphernalia. The swab is pressed onto printed reaction zone for 5 seconds, contacting e-juice sample, solvent, and printed reagents, facilitating a chemical reaction and instant presumptive indication for the presence of Nicotine in the suspect e-juice and/or vaping paraphernalia.,

The description of the present disclosure has been presented for purposes of illustration and description, but is not intended to be exhaustive or limited to the invention in the form disclosed. Many modifications and variations will be apparent to those of ordinary skill in the art. The implementation was chosen and described in order to best explain the principles of the invention, the practical application, and to enable others of ordinary skill in the art to understand the disclosed implementations for various implementations with various modifications as are suited to the particular use contemplated.

The abbreviations used herein have their conventional meanings within the chemical and biological arts.

While various implementations and aspects of the disclosure are shown and described herein, it will be obvious to those skilled in the art that such implementations and aspects are provided by way of example only. Numerous variations, changes, and substitutions will now occur to those skilled in the art without departing from the disclosed implementations. It should be understood that various alternatives to the implementations described herein may be employed.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al. MOLECULAR CLONING, A LABORATORY MANUAL, Cold Springs Harbor Press (Cold Springs Harbor, N Y 1989). Any methods, devices and materials similar or equivalent to those described herein can be used in the practice of this the disclosed implementations. The following definitions are provided to facilitate understanding of certain terms used frequently herein and are not meant to limit the scope of the present disclosure.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B:” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the disclosed implementations. For example, “0.2-5 mg” is a disclosure of 0.2 mg. 0.3 mg. 0.4 mg, 0.5 mg, 0.6 mg etc. up to and including 5.0 mg.

The term “of narcotic(s)” is intended to relate to all suspect residue(s) from commercial and illicit products and formulations in solid or liquid form. thereof. The term “equivalent to about . . . of narcotic, drug, contraband” is intended to relate to a specified volume, concentration, or amount of narcotic provided by a volume, concentration, or mass.

The term “narcotic(s)” is intended to relate to all conventional and classic street drugs of abuse and new and emerging novel designer drugs and drugs of natural plant, bacterial or fungal origin and/or derived from synthetic laboratory procedures. thereof. The term “equivalent to about . . . of narcotic, drug, contraband” is intended to relate to a specified volume, concentration, or amount of narcotic provided by a volume, concentration, or mass.

The term “contraband” is intended to relate to narcotic residue hidden, concealed or otherwise deliberately masked. thereof. The term “equivalent to about . . . of contraband” is intended to relate to a specified volume, concentration, or amount of contraband provided by a volume, concentration, or mass.

As used herein, “salts” or “salt form” or “pharmaceutically accepted salts” may include base addition salts (formed with free carboxyl or other anionic groups) which are derived from inorganic bases such as, for example, sodium, potassium, ammonium, calcium, or ferric hydroxides, and such organic bases as isopropylamine, trimethylamine, 2-ethylamino-ethanol, histidine, procaine, and the like. Such salts are formed as acid addition salts with any free cationic groups and generally are formed with inorganic acids such as, for example, hydrochloric, sulfuric, or phosphoric acids, or organic acids such as acetic, citric, p-toluenesulfonic, methanesulfonic acid, oxalic, tartaric, mandelic, and the like. Salts of the disclosure may include amine salts formed by the protonation of an amino group with inorganic acids such as hydrochloric acid, hydrobromic acid, hydroiodic acid, sulfuric acid, phosphoric acid, and the like. Salts of the disclosure also include amine salts formed by the protonation of an amino group with suitable organic acids, such as p-toluenesulfonic acid, acetic acid, and the like. Additional excipients which are contemplated for use in the practice of the present disclosure are those available to those of ordinary skill in the art, for example, those found in the United States Pharmacopoeia Vol. XXII and National Formulary Vol. XVII. U.S. Pharmacopoeia Convention. Inc., Rockville. Md. (1989). the relevant contents of which is incorporated herein by reference

What is claimed is:

1. A detection kit for identifying the presence of a drug, the kit comprising:
a dry colorimetric reagent and a delivery device containing a solvent or solvent mixture including the solvent;
wherein the delivery device is configured to deliver a portion of the solvent or solvent mixture to a target residue to form a sample residue;
wherein, when at least a portion of the sample residue is brought into contact with the mixture, the mixture undergoes a chemical reaction when the sample residue contains a drug; and
wherein the chemical reaction produces a visible color change that is indicative of the presence of the drug within the sample residue.

2. The detection kit of claim 1, wherein the dry colorimetric reagent comprises bismuth nitrate, potassium iodide, and an acid.

3. The detection kit of claim 2, wherein the acid is an organic acid.

4. The detection kit of claim 3, wherein the organic acid is selected from the group consisting of tartaric acid, ascorbic acid, oxalic acid, toluenesolfonic acid, benzoic acid, citric acid, and any combination thereof.

5. The detection kit of claim 2, wherein the acid is an inorganic acid.

6. The detection kit of any one of the preceding claims, wherein the mixture is configured to undergo chemical reaction with the drug in the form of liquids, gels or solid powders that are pure or admixed with cutting agents.

7. The detection kit of any one of the preceding claims, wherein the drug is at least one of an amphetamine, cocaine, cathinone, heroin, lysergic acid diethylamide, nicotine, a synthetic cannabinoid, and a fentanyl analogue.

8. The detection kit of any one of the preceding claims, further comprising a substrate having the dry colorimetric reagent disposed on at least a portion thereof.

9. The detection kit of claim 8, wherein the at least a portion of the substrate with the dry colorimetric reagent forms a reaction zone, wherein the chemical reaction occurs at the reaction zone.

10. The detection kit of claim 8 or 9, wherein the substrate is formed of at least one of fibers or polymers.

11. The detection kit of claim 8 or 9, wherein the substrate is a paper card, a paper sheet, a synthetic paper, or chromatography paper.

12. The detection kit of any one of the preceding claims, wherein the delivery device includes an absorbent material.

13. The detection kit of any one of the preceding claims, wherein the delivery device is a cotton swab that absorbs the solvent or solvent mixture.

14. The detection kit of any one of the preceding claims, wherein the delivery device is a snap cotton swab that selectively stores the solvent or solvent mixture in a shaft.

15. The detection kit of any one of claims 8-14, wherein the substrate and the delivery device are each enclosed and separated from each other in respective containers.

16. The detection kit of any one of claims 8-14, further comprising a packet that is configured to selectively house the substrate and the delivery device.

17. The detection kid of claim 16, wherein the packet is hermetically sealed.

18. A method of manufacturing a detection kit, the method comprising:
preparing an ink formulation that includes a colorimetric reagent and at least one solvent;
applying, by a printing process, the ink formulation on a least a portion of a substrate; and
drying the ink formulation to form a dry colorimetric reagent on the substrate;
wherein the dry colorimetric reagent is configured to undergo a chemical reaction with a sample residue having a drug that produces a visible color change colorimetric indication.

19. The method of claim 18, wherein the dry colorimetric reagent comprises bismuth nitrate, potassium iodide, and an acid.

20. The method of claim 19, wherein the acid is an organic acid.

21. The method of claim 20, wherein the organic acid is selected from the group consisting of tartaric acid, ascorbic acid, oxalic acid, toluenesolfonic acid, benzoic acid, citric acid, and any combination thereof.

22. The method of claim 19, wherein the acid is an inorganic acid.

23. The method of any one of claims 18-22, wherein the dry colorimetric reagent on the substrate forms a reaction zone, wherein the chemical reaction occurs at the reaction zone.

24. The method of any one of claims 18-23, further comprising storing a solvent or solvent mixture including the solvent within a delivery device, wherein the delivery device is configured to deliver a portion of the solvent or solvent mixture to a target residue to form the sample residue.

25. The method of claim 24, wherein storing the solvent or solvent mixture comprises, dipping the delivery device into a solution that includes the solvent or solvent mixture.

26. The method of claim 24 or 25, wherein the solvent mixture comprises a denatured alcohol and water in a ratio of 50:50, wherein the denatured alcohol is present in the solvent mixture at a volume of about 0.05 mL to about 0.2 mL.

27. The method of any one of claims 18-26, wherein preparing the ink formulation comprises,
dissolving a first reagent of the colorimetric reagent and the at least one acid into a first solution;
dissolving a second reagent of the colorimetric reagent in a second solution; and
mixing the first and second solutions together to form the ink formulation.

28. The method of claim 27, wherein the first solution comprises denatured alcohol, water, and glycerol.

29. The method of claim 27 or 28, wherein the second solution comprises water.

30. The method of any one of claims 18-29, wherein the substrate is a bibulous carrier material.

31. The method of any one of claims 18-29, wherein the substrate is a cellulose chromatography paper.

32. The method of any one of claims 18-31, wherein the printing process is one of spray printing, dip printing, flexography, offset screen printing, lithography, digital printing, or gravure printing.

33. The method of any one of claims 18-31, wherein the printing process is a flexographic printing process.
